(19) United States
(12) Patent Application Publication
(10) Pub. No.: US 2010/0317945 A1
(43) Pub. Date: Dec. 16, 2010

(54) CUFF FOR DETERMINING A PHYSIOLOGICAL PARAMETER

(76) Inventors: Olaf Schraa, Amsterdam (NL); Bob Schraa, Amsterdam (NL); Jos J.G.M. Settels, De Hoef (NL)

Correspondence Address: PATENT GROUP;C/O DLA PIPER US LLP, 203 N. LASALLE ST., SUITE 1900, CHICAGO, IL, 60601, US

(21) Appl. No.: 12/669,895

(22) PCT Filed: Jul. 20, 2007
(86) PCT No.: PCT/NL2007/050361

§ 371 Date: Aug. 31, 2010

(51) Int. Cl. A61B 5/1455

(52) U.S. Cl. 600/324

(57) ABSTRACT

The invention relates to a cuff (20) for determining a physiological parameter, said cuff comprising a photoplethysmograph arranged with an emitter (6) for emitting a radiation in a direction of a tissue to be investigated, a detector (5) for detecting the radiation from the tissue and an inflatable bladder (8) for transferring pressure to the tissue, said inflatable bladder comprising a back-layer (8″) and a top-layer (8′), wherein the top-layer is conceived to be brought into contact with the tissue, the top-layer being substantially more flexible than the back-layer. The invention further relates to a measurement system.

### FIELD OF THE INVENTION

The invention relates to a cuff for determining a physiological parameter.

### BACKGROUND OF THE INVENTION

An embodiment of a cuff as is set forth in the opening paragraph is known from U.S. Pat. No. 4,726,382. The known cuff comprises an inflatable bladder formed from a thin flexible, translucent material. The inflatable bladder is connected to a tube enabling a suitable inflation and deflation of the inflatable bladder. In the known cuff the inflatable bladder is the innermost component of the cuff. The inflatable bladder is manufactured from two strips of film being heat sealed together about their periphery thereby forming a cavity. The known cuff is arranged to be fit about a person's finger. For this purpose the inflatable bladder is provided with a back-layer facing outwards from a tissue and a top-layer facing the tissue and being conceived to be brought into contact with the tissue. In order to implement a measurement of a physiological parameter, the known cuff comprises a photoplethysmograph.

In the inflatable bladder of the known cuff openings are provided so that a suitable light source and a light detector can be mounted therein. The inflatable bladder is mounted on a flexible printed circuit. The flexible circuit is used to feed components of the cuff and to supply measurement signals from the cuff to a suitable data processing unit.

It is a disadvantage of the known cuff in that both the back-layer and the top-layer of the inflatable bladder deform to some extent due to applied cuff pressure. This may result in inaccurate reading of physiological parameters such as blood pressure of the cuff due to a changing angle of reflection for a reflective set-up, or a displacement between, for example, the emitter and detector for a transmissive set-up.

### SUMMARY OF THE INVENTION

An object of the invention may be to provide a cuff with improved operational characteristics. A further object may be to provide a cuff which can be manufactured in a simplified way. A still further object may be to provide an improvement of or an alternative for a cuff known from the prior art.

According to a first aspect of the invention the cuff may comprise:

a photoplethysmograph arranged with

an emitter for emitting a radiation in a direction of a tissue to be investigated;

a detector for detecting the radiation from the tissue; and

an inflatable bladder for transferring pressure to the tissue, said inflatable bladder comprising a back-layer and a top-layer, wherein the top-layer is conceived to be brought in contact with the tissue, the top-layer being substantially more flexible than the back-layer.

In accordance with this technical measure only the top-layer of the inflatable bladder undergoes deformation under application of a suitable internal cuff pressure. Thus, the internal pressure may be substantially applied to the tissue and substantially not towards internal components of the cuff by the back-layer. Because the back-layer substantially does not undergo any deformation, an emitter—detector, arranged beyond the back-layer, substantially preserve their respective geometry for different operational conditions, like different shapes of objects conceived to be received by the cuff. This substantially improves accuracy of the measurement of a physiological parameter, for example an arterial blood pressure and/or the blood flow and/or the blood oxygenation and may provide a great versatility for the cuff.

Preferably, the back-layer and the top-layer are implemented from respective materials, having comparable thicknesses. In this case a material of the top-layer refers to a substantially elastic material, whereas a material of the back-layer refers to a substantially not-elastic material. It is noted that the term “elastic material” comprises at least any material having a tensile stress at 50% strain of less than 5 MPa. The term non-elastic material comprises at least any material having a yield strength of more than 50 MPa. Presented values hold for a measurement technique of, for example ASTM, DIN or NEN. Suitable materials for the top-layer comprise but are not limited to polyurethanes. Suitable materials of the back-layer can be chosen from but not limited to polyvinyl chloride. It will be appreciated that the back-layer may comprise a thickened layer of material or a multilayer structure. In this case the back-layer may be manufactured from the same material as the top-layer, however having an increased thickness. Alternatively, the back-layer may have substantially the same thickness as the top-layer, but may be enforced with a further material, like a mesh or wiring for reducing its flexibility in use. It is noted that suitable embodiments of the emitter comprise a light source, for example a red LED source, or an infrared LED source.

Photoplethysmographs are known per se. An operational principle of the photoplethysmograph is based on the fact that with each cardiac cycle the heart pumps blood to the periphery of the body.

A change in volume of the arteries or arterioles caused by the pressure pulse of the systolic wave is detected by illuminating the skin with a suitable light, notably emitted from a Light Emitting Diode (LED) and then measuring the amount of light either transmitted or reflected to a suitable detector, notably a photodiode. Each cardiac cycle appears as a peak in a signal from the photoplethysmograph. The shape of a signal waveform from the photoplethysmograph differs from subject to subject, and varies with a location and a manner in which the cuff is attached to the tissue. It is noted that the photoplethysmograph can be attached to a great plurality of areas on the human body, for example on a finger, on an ear, in a nostril, on the temples of the head. The photoplethysmograph may even be arranged in a body cavity.

In an embodiment of a cuff, a flexible printed circuit may be provided for feeding the photoplethysmograph and for acquiring measurement data from the detector, whereby the back-layer may be arranged on the flexible printed circuit. In case the back-layer is adhered to the flexible printed circuit the latter may serve as a substantially stable foundation, decreasing a risk of undesired deformation of the back-layer in use. This feature further improves accuracy of a measurement. The back-layer of the inflatable bladder may be directly attached to the flexible circuit thereby reducing a number of components of the cuff. In a further embodiment of the cuff the top-layer may be mounted directly airtight to the flexible printed circuit totally eliminating the back layer. This may reduce the manufacturing costs accordingly.

In an embodiment of the cuff the top-layer can be arranged to provide an electrical insulation between the tissue and electrical components of the cuff. In accordance with this technical measure the top-layer advantageously has an additional function which may obviate a necessity for a specific anti-dust or anti-sweat protective layer. This has an advantage in that an additional decrease in a number of functional elements of the cuff is provided. The top layer may further be arranged to protect suitable electrical components of the cuff from contamination, like dirt and/or sweat.

In a still further embodiment of the cuff, the top-layer at least partially comprises a biocompatible material. In particular, a surface of the top-layer conceived to contact the tissue may be provided with or may consist of a bio-compatible material. This has an advantage that a risk of irritating the tissue is minimized, which is advantageous for a long-term monitoring. Preferably, the top-layer extends further than a surface of the inflatable bladder conceived to come into contact with the tissue in use. Due to this feature other layers of the cuff do not have to be manufactured from a biocompatible material, reducing the production costs or providing a wider choice of materials which can be employed. Also the electrical components need not to be made of biocompatible materials if the top layer is arranged over it. The material of the top-layer preferably comprises at least a part which is transparent to the radiation emanated from the emitter of the photoplethysmograph. However, it is also possible that the material of the top-layer comprises at least a part which is reflective to the said radiation. Preferably, the radiation is in the infra-red range.

In a further embodiment of the cuff the back-layer may be provided with cut-away areas for transmitting incident radiation from the emitter to the tissue and/or for transmitting the radiation from the tissue to the detector. It should be understood that the radiation from the tissue refers to either a transmitted radiation or a reflected radiation. Preferably, the emitter and the detector are arranged in the cuff so that respective external surfaces of the emitter and the detector lie substantially in a plane of the back-layer. The emitter and the detector can be mounted directly in the cut-away areas allowing easy and reliable automatic attachment, for example, soldering, of the cuff components. For this purpose the flexible printed circuit may be provided with similar cut-away areas which cooperate with the cut-away areas of the back-layer. Such arrangement of the emitter and the detector can have an additional advantage in that a height of the emitter and the detector with respect to a surface of the flexible printed circuit facing the back-layer can be adjusted, causing a reduction of a pressure applied by the emitter and the detector to the tissue, which is more comfortable in use. Simultaneously, the emitter and the detector are arranged in the cuff so that they substantially contact the tissue in use for enabling optimal coupling of the radiation with the tissue.

The material of the flexible printed circuit may be substantially flexible having low elasticity. It is found to be advantageous to provide the flexible printed circuit as a somewhat stiff structure, which may be bendable with a certain radius. This feature may be used to improve stiffness of a flap, which is a part of the cuff wrapped around a body portion, notably a finger, outside the inflatable bladder. The flexible circuit may be made of an infrared opaque material, alternatively, the flexible circuit may be reflective to infrared radiation.

In a further embodiment of the cuff according to the invention, a surface of the flexible printed circuit conceived to face the tissue in use comprises an electrically conductive coating. In case the emitter comprises a light source the coating may also be optically opaque and reflective. In accordance with this technical measure the light impinging on the coating will be reflected back towards the tissue improving signal-to-noise ratio. In addition proper electrical shielding is enabled. In addition, the opacity of the flexible circuit material advantageously prevents the detector of the photoplethysmograph from interference of ambient light. It is noted that in this case a usually envisaged protective layer for covering suitable metallic traces of the flexible printed circuit can be left out on the inner surface of the flexible printed circuit, which may further reduce manufacturing costs of the cuff and improves reflectivity and flexibility.

In a further embodiment of the cuff, the flexible printed circuit may be shaped with a tail-end for relieving fastening strain in use. Preferably, strain of an electric cable of the cuff and/or strain of an air tube of the cuff is relieved. This feature advantageously improves endurance of the cuff, in particular when the cuff is conceived to be repetitively fastened and removed.

In a further embodiment of the cuff, the flexible printed circuit may be arranged with an identifying unit and/or a signal processing unit for processing the measurement data. Suitable known per se circuits may be used for performing signal conditioning, like filtering, and amplifying. The identifying unit can, for example, be used to determine which size cuff is attached. This information can be used, for example, for the optimisation of servo parameters.

In a further embodiment of the cuff, the flexible printed circuit may comprise metal traces to solder positions being oriented substantially axially to a bending curve of the flexible print circuit. Preferably copper is used for this purpose. Due to this technical measure a chance of fracturing the traces upon bending is minimized.

In a still further embodiment of the cuff, the emitter may be arranged for emitting a focused beam of radiation. It is found to be advantageous to provide the focused radiation beam for avoiding pulsations or lack of pulsations from areas distant to a central location underneath the cuff, as such pulsations may cause an erroneous reading of the detector; lack of pulsations may compromise signal strength. Preferably, the emitter comprises a built-in opaque shield. This feature has an advantage in that stray light is minimized. The emitter may preferably be arranged to emit radiation with a wavelength in a range of 660-1000 nm. Preferably, the detector comprises a photodiode arranged to be sensitive at least to a portion of radiation emanating from the emitter.

In a still further embodiment of the cuff according to the invention, the cuff further may comprise means for preventing blockage of an airflow in an air tube communicating with the inflatable bladder. For example, the air tube may be cut at an angle less than 90 degrees and may be mounted in the inflatable bladder with the longest projection towards the top-layer.

These and other aspects of the invention will be discussed in more detail with reference to drawings, wherein like reference signs refer to like elements.

### BRIEF DESCRIPTION OF THE DRAWINGS

FIG. 1 presents a schematic elevated view of a cuff according to the invention.

FIG. 2 presents a schematic view of the cuff of FIG. 1 in a flat condition.

FIG. 3 presents a schematic view of the cuff of FIG. 1 in use.

FIG. 4 presents a schematic view of a measurement system according to the invention.

### DETAILED DESCRIPTION

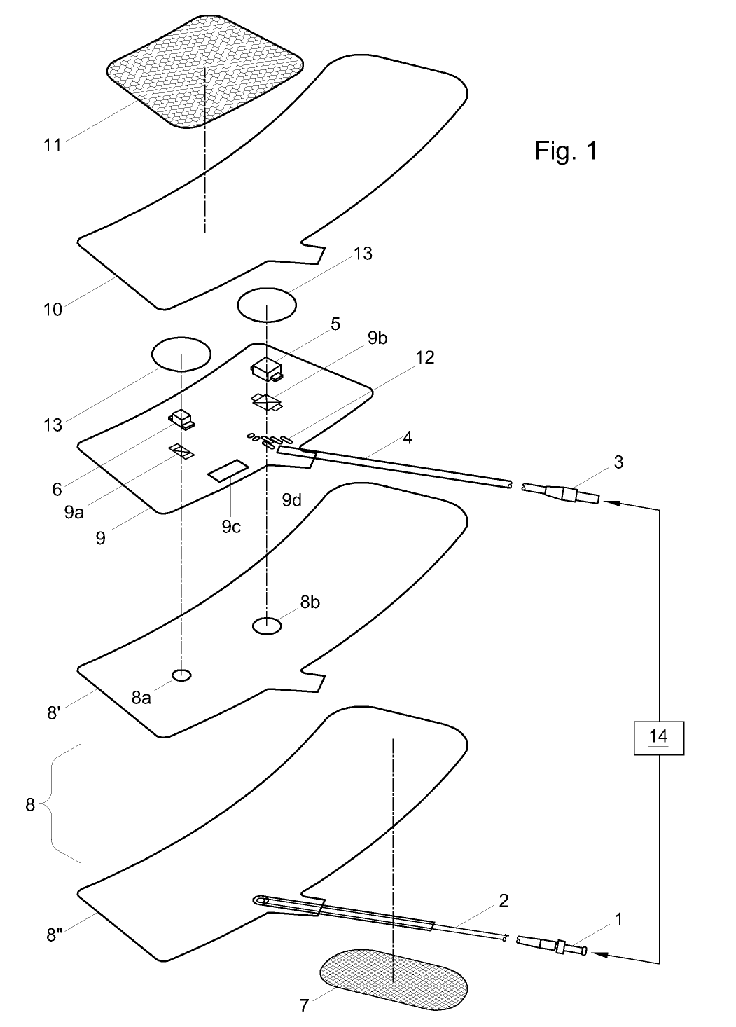

It will be appreciated that the drawings, setting out some aspects of the cuff according to the invention, are not limitative. FIG. 1 presents a schematic elevated view of a cuff according to the invention. The cuff comprises an inflatable bladder 8 provided with an air supply channel 2 for inflating the bladder and for evacuating it. For this purpose the air supply channel 2 may comprise a suitable fitting 1 for connecting to a pump, notably a gas pump. The inflatable bladder 8 comprises a top-layer 8″ conceived to be brought into contact with a portion of a person, notably with a finger of the person. The inflatable bladder further comprises a back-layer 8′ attached to a flexible printed circuit 9. The top-layer is at least more elastic than the back-layer. Elastic should be understood as at least but not limited to any material having a tensile stress at 50% strain of less than 5 MPa. Preferably, the back-layer is substantially or entirely not-elastic. The term non-elastic material refers to at least any material having a yield strength of more than 50 MPa. Presented values hold for a measurement technique of ASTM, DIN or NEN. It is noted that the back-layer is preferably directly attached to the flexible printed circuit 9 without using any additional adhesive inter-layers for reducing a number of components of the cuff. The top-layer 8″ may comprise a substantially flexible and elastic material, like polyurethane. The back-layer 8′ may comprise a substantially flexible non-elastic material, like polyvinyl chloride. The back-layer 8′ may be attached to the top-layer 8″ by any suitable technique, preferably a sealing method is used. The back-layer, the flexible printed circuit 9 or the label may be non-elastic. Or two or all of these, whereas the non-elasticity may be provided for or enhanced by other means, such as non-elastic wires incorporated in a longitudinal direction L of the cuff.

The inflatable bladder 8 may further comprise cut-away areas 8a and 8b, wherein a light source 6, such as a light emitting device (LED) and a light detector 5 are positioned, respectively. The back-layer 8′ of the inflatable bladder 8 can be attached to the flexible printed circuit 9, which comprises corresponding cut-away areas 9a, 9b for accommodating the light source 6 and the light detector 5. The flexible printed circuit 9 may further comprise suitable blockers 13 for shielding the light source 6 and the detector 5 from interference with other light sources or detectors. Preferably, the blockers 13 comprise opaque flexible material. A signal from the light detector 5 is picked up by one or more suitable electronic components (not shown) of the flexible printed circuit 9. The flexible printed circuit 9 is electrically connectable to a cable 4 provided with a suitable electric connector 3. It is possible that the cable 4 and the air supply 2 are housed in a joint housing having a single outside connector 14. The flexible printed circuit may comprise an identifying unit 12. Preferably, the flexible printed circuit 9 further comprises a module 9c for processing the signal from the detector 5. Suitable signal processing steps performed by the computers and/or a processing unit to be connected to the cable 4 may comprise a filtering, amplification, or the like.

A cuff 20 according to the invention may further comprise a sticker 10, which may comprise or form a suitable label. Fastening means, such as loop 11 and hook 7 material, for example Velcro® may be arranged on the cuff 20 to fasten the cuff about a portion of a recipient, for example around a finger of a person. Preferably, the top-layer 8″ is manufactured from a biocompatible material and extends substantially over the same length as the back-layer 8′ or the sticker 10. Due to the fact that only the biocompatible top-layer is in contact with the tissue, the label or the back-layer does not have to be manufactured from a biocompatible material reducing the production costs of the cuff.

Preferably, a surface of the flexible printed circuit conceived to face the tissue in use comprises a coating, preferably a coating which is electrically conductive and optically opaque and/or reflective. This can have an effect that the light that may be reflected from the tissue and impinges on the coating, will be reflected back towards the tissue, improving signal-to-noise ratio of the measurement signal. In addition, such coating may enable proper electrical shielding of the components of the flexible printed circuit. The opacity of the flexible circuit material may advantageously prevent the detector of the photoplethysmograph from interference of ambient light with the photoplethysmograph, which also contributes to an improvement of the signal-to-noise ratio of the cuff according to the invention. Preferably, metal traces, notably copper traces, are used in the flexible printed circuit to connect the electrical cable 4 to the components of the flexible printed circuit, which makes wiring redundant, further decreasing manufacturing costs of the cuff according to the invention.

In a further embodiment of the cuff, the flexible printed circuit is shaped with a tail-end 9d, such as a projection, for relieving fastening strain applied to the cabling 4 and the air duct 2 in use. This improves durability of the cuff, particularly when it is conceived to be repetitively fastened and removed.

The cuff 20 is conceived to be arranged on a suitable portion of a body, for example around a finger, on an ear or the temples of the head, in a nostril, or in a body cavity. The cuff 20 comprises a photoplethysmograph arranged with an emitter 6, for example a LED or an infra-red source, and a light detector 5, for example a photodiode. This emitter-receiver pair is used to determine a blood flow in said portion of the body. In case a transmissive set-up is used, a light emitting diode may be used to transmit light through the skin. The detector 5 picks up the transmitted signal, which can then be analyzed using suitable signal processing techniques. A signal from the photoplethysmograph comprises a pulse wave corresponding to changes in blood volume in the arteries or capillaries receiving the light from the emitter 6. Changes in blood volume produce changes in optical absorption of the emitted light. The light transmitted through the tissue can be highly scattered or absorbed depending on the tissue. The detector, which is positioned on the surface of the skin, can detect the transmission of waves from various depths and from highly absorbing or weakly absorbing tissues. Regardless of the absorbency of the tissues and skin, it is generally assumed that the amount of light absorbed and/or reflected by these tissues will remain constant. With this assumption in mind, it can then be assumed that the only change in the absorption or reflection of the transmitted light will be from the increase or decrease of the blood volume in the arteries and capillaries. The measured volume change is actually an average of all of the arteries and capillaries in the space being irradiated. For a transmissive set-up infrared radiation may be chosen for the emitter 6 because infrared is well absorbed by blood and very weakly absorbed by other tissues and fluids in the body. This means that the blood volume changes can be very easily observed. Preferably, for detecting the signal an infrared receiver is chosen, sensitivity of which matches at least partially the spectrum of the infrared emitter. It may also be beneficial to use an infrared emitter because changes in blood oxygen content are very prominent in the visible light region. Due to the fact that the inflatable bladder comprises a top-layer which is more elastic than the back-layer, measurements of a physiological parameter such as blood pressure are obtained with improved accuracy, because less or no distortion of a light path between the light source and the light detector is caused by a deformation of the back-layer.

The placement of the emitter and receiver on the body is also an important aspect of the photoplethysmograph. It may be chosen to position the photoplethysmograph at an earlobe or at the fingers, because of the consistency of the tissues. These areas on the body can also be held relatively still to reduce motion artifact which would otherwise distort the output signal. Still another applicable area may be the nasal septum. However, different positions are possible.

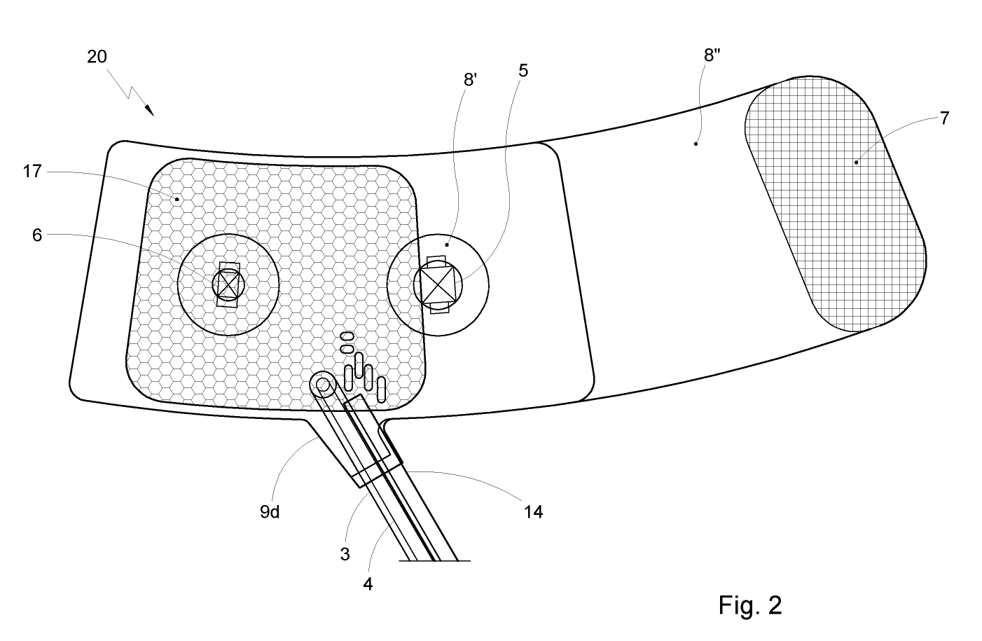

FIG. 2 presents a schematic view of the cuff of FIG. 1 in a flat condition. The cuff 20 is shown in a top view, revealing a substantially transparent top-layer 8″ where through a back-layer 8′ is seen provided with cut-way areas for accommodating respective parts of the photoplethysmograph, notably the light source 6 and the light detector 5. The cuff 20 can be inflated using air supply duct 2. Measurement data from the photoplethysmograph can be collected using a suitable electric cable 4. The electric cable 4 and the air supply channel 2 may be arranged in a suitable tube 14 which is attached to the cuff 20 by means of a tail portion 9d. The cuff 20 may be conceived to be wrapped around a body portion, for example a finger, in this case the cuff 20 may comprise fastening means, for example hooks 7 cooperating with loops 17.

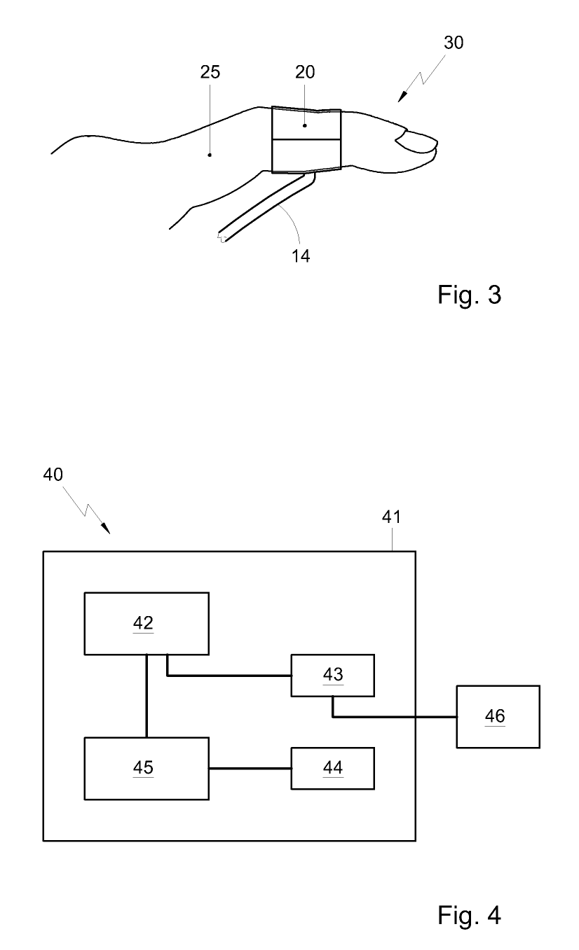

FIG. 3 presents a schematic view 30 of the cuff of FIG. 1 in use, wherein the cuff 20 is wound about a finger 25 of a person under investigation. It is noted that especially when the top-layer of the cuff 20 comprises a bio-compatible material, the cuff 20 may be used for substantially prolonged period of time on the person, for example for purposes of durable monitoring of a vital sign, notably of pressure and/or cardiac output.

FIG. 4 presents a schematic view of a measurement system according to the invention. The system 40 comprises a processor 41 and a measurement unit 46, which may preferably comprise the cuff for carrying measurements of the arterial pressure waveform, as is set forth with reference to FIG. 1. Data collected by the measurement unit 46 is provided to the input 43 of the processor 41. It is noted that using the measurement unit 46, for example the cuff as is discussed with reference to FIG. 1, a plurality of useful signals may be acquired. For example, plethysmogram and blood pressure data may be used for determining in non-invasive way additional information, for example using suitable models applicable to said data. For example, cardiac output can be determined non-invasively using arterial data provided by the measurement unit 46. The processor 41 further comprises storage 42 for storing a suitable model, for example a non-linear model approximating a relation between an aortic cross-section and applied pressure. Preferably, for the non-linear model an arctangent model is selected.

The aortic mechanical properties approximate a response of the internal cross-sectional area of the aorta to an increase in pressure by an arctangent:

A(p)=Am(0.5+(1/π)arctan((p−p0)/p1))

in which A the cross-sectional area in cm²;
Am the maximal area at very high pressure;
p0 indicating the inflexion point of a pressure curve;
p1 indicating a halfwidth of a pressure pulse.

The processor 41 further comprises a computing unit 45 arranged for computing a compliance and/or an impedance of an aortic portion from the acquired arterial pressure data. For obtaining the compliance or the impedance the non-linear model, notably the arctangent model, is differentiated. The thus obtained value of the compliance or impedance may be incorporated into per se known pulse contour method, like for example the Waterhammer model or Windkessel model, for determining the beat-to-beat stroke volume and/or cardiac output based on the measured arterial pressure data, notably the waveform.

Windkessel model is a linear model pulse contour model that describes a relation between a stroke volume Vs, aortic compliance and characteristics of the waveform of arterial pressure.

Vs=C(p2−p1)(1+As/Ad)

with Vs—a stroke volume, C—an aortic compliance, defined as dV/dP, p2—a pressure at a dicrotic notch, p1 the diastolic pressure, As the integrated area under the systolic portion of the blood pressure curve, and Ad similarly the diastolic area. The dicrotic notch is a pulse that precedes a dicrotic wave, it being a pulse sequence comprising a double-beat sequence wherein a second beat is weaker than a first beat.

Waterhammer model is another linear pulse contour model that describes a relation between impedance Zc, density of blood, aortic cross-sectional area and aortic compliance:

Zc=√(r/(A C′))

with r the density of blood, A the aortic cross-sectional area and C′ the compliance per unit length.

It is noted that the cardiac output equals the stroke volume multiplied by a heart beat frequency. In this way a system is provided for determining beat-to-beat stroke volume and/or cardiac output based on the measurement of an arterial pressure waveform with increased accuracy compared to prior art.

It is found to be advantageous to provide a-priori tabulated relationships between theses parameters and patient characteristics, like age and gender. The computing unit 45 may be arranged to use these tabulations to calculate the compliance data from the arctangent model, yielding:

C(p)=Cm/(1+((p−p0)/p1)²)

with

Cm=Am/(πp1),

wherein

C(p) is a pressure-dependent compliance;

Cm—is a maximum compliance or the aortic portion.

The computing means 45 is then arranged to incorporate the calculated value of the compliance in a linear model to calculate the beat-to beat stroke volume and/or cardiac output.

While specific embodiments have been described above, it will be appreciated that the invention may be practiced otherwise than as described. The descriptions above are intended to be illustrative, not limiting. Thus, it will be apparent to one skilled in the art that modifications may be made to the invention as described in the foregoing without departing from the scope of the claims set out below.

1. A cuff for determining a physiological parameter, comprising:
a photoplethysmograph arranged with
an emitter for emitting a radiation in a direction of a tissue to be investigated;
a detector for detecting the radiation from the tissue; and
an inflatable bladder for transferring pressure to the tissue, said inflatable bladder comprising a back-layer and a top-layer,
wherein the top-layer is conceived to be brought into contact with the tissue, and wherein the back-layer comprises a flexible substantially non-elastic material which does not undergo deformation a suitable internal cuff pressure and the top layer comprises a flexible substantially elastic material which undergoes deformation on under application of the suitable internal cuff pressure.

2. (canceled)

3. A cuff according to claim 1, wherein the top-layer is arranged to provide an electrical insulation between the tissue and electrical components of the cuff.

4. A cuff according to claim 1, wherein the top-layer is arranged to protect electrical components of the cuff from contamination.

5. A cuff according to claim 1 wherein the top-layer is at least partially manufactured from a biocompatible material.

6. A cuff according to claim 5, wherein the top-layer extends further than an area of the inflatable bladder conceived to come into contact with the tissue in use.

7. A cuff according to clam 1, wherein the back-layer is provided with cut-away areas for transmitting the radiation from the emitter to a tissue or for transmitting a further radiation from the tissue to the detector.

8. (canceled)

9. A cuff according to claim 1, wherein the height of the optical components (LED and photo diode) is adjustable.

10. (canceled)

11. (canceled)

12. (canceled)

13. (canceled)

14. A cuff according to claim 1, wherein the emitter is arranged for emitting a focused beam of radiation.

15. A cuff according to claim 1, wherein the emitter comprises a built-in opaque shield.

16. A cuff according to claim 1, wherein the emitter is arranged to emit radiation with a wavelength in a range of 660 to 1000 nm.

17. A cuff according to claim 1, wherein the detector comprises a photodiode arranged to be sensitive for a radiation with a wavelength matching at least part of a spectrum of the emitter.

18. A cuff according to claim 1, further comprising:
an air tube communicating with the inflatable bladder;
means for preventing blockage of an airflow in the air tube.

19. A cuff according to claim 18, wherein the air tube is cut at an angle less than 90 degrees and is mounted in the inflatable bladder with the longest projection towards the top-layer.

20. (canceled)

21. A measurement system comprising a cuff according to claim 1 and a data processing unit.

22. A measurement system according to claim 21, wherein the data processing unit is arranged to:
compute a compliance or impedance in dependence of at least one measurement of arterial pressure data and patient data using a non-linear model;
use said compliance or impedance in a pulse contour method for determining the beat-to-beat stroke volume or cardiac output based on the measured arterial pressure data.

23. (canceled)

24. (canceled)

25. (canceled)
